(19) United States
(12) Patent Application Publication
(10) Pub. No.: US 2005/0259779 A1
(43) Pub. Date: Nov. 24, 2005

(54) Biomolecular contrast agents for therapy optimization in radiation therapy with proton or ion beams

(75) Inventors: Klaus Abraham-Fuchs, Erlangen (DE); Michael Moritz, Mistlegau (DE)

Correspondence Address: Siemens Corporation;Attn: Elsa Keller, Legal Administrator, Intellectual Property Department, 170 Wood Avenue South, Iselin, NJ, 08830, US

(73) Assignee: Siemens Aktiengesellschaft

(21) Appl. No.: 10/848,871

(22) Filed: May 18, 2004

(51) Int. Cl. G01T 1/00; A61N 1/00

(52) U.S. Cl. 378/2; 607/2

(57) ABSTRACT

Bio-molecular contrast agents (BMCA) can be used for optimizing radiation dosage, energy and/ or duration in order to achieve on-line, real-time therapy optimization. The BMCA signals during treatment are compared with expected values to determine what, if any, therapy optimization is needed.

### CROSS-REFERENCE TO RELATED APPLICATIONS

This application is related to 1) a patent application entitled “Biomolecular Contrast Agents For Therapy Success And Dose Monitoring In Radiation Therapy With Proton Or Ion Beams” bearing attorney docket number 2004P01914US, filed concurrently herewith, and incorporated by reference herein; 2) a patent application entitled “Biomolecular Contrast Agents For Therapy Control In Radiation Therapy With Proton Or Ion Beams” bearing attorney docket number 2003P019082US, filed concurrently herewith and incorporated by reference herein; and 3) a patent application entitled “Biomolecular Contrast Agents With Multiple Signal Variance For Therapy Planning And Control In Radiation Therapy With Proton Or Ion Beams” bearing attorney docket number 2003P19081US, filed concurrently herewith and incorporated by reference herein.

### BACKGROUND

1. Field of the Invention

This invention relates generally to the art of radiation therapy and diagnostic imaging. More specifically, the invention relates to the use of contrast agents in therapy planning and treatment involved in radiation therapy.

2. Related Art

In the treatment of cancer and other diseases, therapeutic measures such as particle beam therapy are commonly employed. In particle beam therapy, a beam (or beams) of radiation in the form of electrons, or photons, or more recently, protons, is delivered to a tumor or other target tissue. The dosage of radiation delivered is intended to destroy the tumorous cells or tissues.

It is state of the art today that medical imaging techniques such as CT (Computed Tomography), MR (Magnetic Resonance), PET (Positron Emission Tomography), optical imaging (ultraviolet/infrared/visible) or ultrasound are used to visualize the target region (most often a tumor) for particle beam therapy. Yet, the medical imaging techniques used for this purpose in many cases cannot reliably differentiate between malign tumors and benign tumors, and in particular are not well suited to visualize exactly the borderline between healthy tissue and malign tumors. Thus the therapy control methods today are based on non-optimal medical images, and as a consequence, for the sake of a successful destruction of the tumor, the volume to be irradiated usually is chosen larger than absolutely necessary thereby damaging healthy tissue in the process. Exact positioning and dosage is especially critical in therapies that use proton beams, where the energy is highly concentrated in particular locations due to the well-know Bragg Peak phenomenon.

Additionally, it happens in many cases that the images used for therapy planning do not exactly show the location of the target tissue for irradiation during the therapy session, for example because the patient is not positioned exactly in the same way during the imaging and the therapy session, or because the filling of the intestinal tract is different in both sessions, and thus organs are shifted. The composition and relative thickness of fatty tissue, fluids, muscle, and connective tissue in the beam pathway needs to be known, and unfortunately, can change after therapy planning. Recently, artificial or anatomical landmarks are used to control the position of the target tissue.

One solution that has been used recently in some imaging techniques is the introduction of “contrast agents” which enhance the image quality achieved during imaging. To provide diagnostic data, the contrast agent must interfere with the wavelength of radiation used in the imaging, alter the physical properties of the tissue/cell to yield an altered signal or provide the source of radiation itself (as in the case of radio-pharmaceuticals). Contrast agents are introduced into the body of the patient in either a non-specific or targeted manner. Non-specific contrast agents diffuse throughout the body such as through the vascular system prior to being metabolized or excreted. Non-specific contrast agents may for instance be distributed through the bloodstream and provide contrast for a tumor with increased vascularization and thus increased blood uptake. Targeted agents bind to or have a specific physical/chemical affinity for particular types of cells, tissues, organs or body compartments, and thus can be more reliable in identifying the correct regions of interest.

Several different targeted contrast agents which bind to particular tissue and then exhibit signal changes based upon state changes in tissues (which are then imaged) are disclosed in international patent application WO 99/17809, entitled “Contrast-Enhanced Diagnostic Imaging Method for Monitoring Interventional Therapies”.

In particular, the parameters of therapy that are planned for can often not be guaranteed to succeed nor be accurate due to changes in tissue state, position and surroundings. The methods used today in planning therapy and optimizing therapy in real-time during the therapy session, are sub-optimal and need to be improved.

### BRIEF DESCRIPTION OF THE DRAWINGS

FIG. 1 illustrates therapy optimization using BMCA according to one embodiment of the invention.

FIG. 2 illustrates a system utilizing one or more embodiments of the invention.

### DETAILED DESCRIPTION OF THE INVENTION

In various aspects of the invention, bio-molecular contrast agents (BMCAS) are introduced into a patient for the purpose of radiation therapy planning and treatment. “BMCA”, as the term is used in describing this invention, are at least partially organic contrast agents which have the following properties: 1) they bind to target tissue, cells, and organs, and/or (2) react with metabolic products of the target tissue, cells, and organs by means of highly specific biochemical reactions (such as body-anti-body mechanisms). This yields an improved highly precise image of the target region for irradiation. In some embodiments, the invention also uses BMCA that are designed to have certain signal-giving properties as well as having a binding or reactive function. The reactive function can also activate the signal-giving property of the BMCA. These mechanisms help to ensure that the signals used for therapy planning, monitoring and control originate only from the target tissue.

For instance, fluorescent BMCAs, such as the ones described in U.S. Pat. No. 6,083,486, can be used in conjunction with a medical optical imager, like an optical tomograph or a diaphanoscope. As illustrated by the invention such BMCAs and other BMCAs can be adapted for use in therapy planning and real-time, on-line therapy control. One advantage of such BMCAs over conventional contrast agents is that the BMCAs stay immobilized for a longer period within the target tissue, due to the highly specific and stable binding reaction. Thus BMCAs are available for a longer time period to observe/monitor the target region than are conventional contrast agents.

BMCAs can also be designed or selected such that their signal-giving property diminishes when the BMCA interacts with the particle beam. The BMCA can thus be “inactivated” (with respect to its signal-giving property) through irradiation with a particle beam of enough energy. For instance, a fluorescent contrast agent may be inactivated by destroying the fluorescence property of the BMCA which would involve breaking of the functional covalent C—C and/or C—H bindings of the BMCA through irradiation. In some embodiments of the invention, the beam energy, or respectively the irradiation dose, needed to inactivate the signal-giving property of the BMCA is roughly the same energy or dose as needed for successful medical treatment of the target tissue.

In this way, two types of information can be derived from the BMCA: the presence of the BMCA through specific binding indicates the target region for treatment while subsequent diminishing of the signal by destroyed signal-giving properties of the BMCA through the particle beam indicate that the target region has successfully been treated with the particle beam.

It is especially advantageous for the purpose of therapy optimization if the BMCA is designed such that the irradiation dose necessary the BMCA is designed such that the irradiation dose necessary to inactivate the BMCA corresponds roughly to the dose necessary to destroy DNA material in the target. Destruction of DNA material is one of the most important known mechanisms in the destruction of tumors through particle irradiation. In such a case, it can be assumed that the decrease of signal from the BMCA by interaction with particle beam is proportional to the degree of destruction of the tumor. To achieve this, in accordance with the invention, the BMCA is designed such that, in order to inactivate the signal-giving property of the BMCA, the destruction of one or more functional covalent C—C and/or C—H bindings (in the DNA) is necessary.

BMCA include small molecules and preferably bio-molecules with an affinity or reactivity with the target tissue. The affinity to bind or reactivity can be dependent on tissue state or tissue type or both. Bio-molecules are typically biologically derived or synthesized from naturally occurring elements such as amino acids, peptides, nucleotides and so on. Examples include receptor ligands, saccharides, lipids, nucleic acids, proteins, naturally occurring or genetically engineered anti-bodies. BMCA include those bio-molecules which can bind to proteins in plasma, in the fluid between cells, or in the space between cells. BMCA also includes dyes and other signal generating compounds, as desired. The difference in binding affinity of one bio-molecule versus another can have an effect in the signals that are ultimately received from the BMCA and in the accuracy of the binding to the target tissues. Thus, the specific nature and structure of the BMCA selected for the purpose of therapy control will depend upon which tissue or tissue component is to be bound. The binding sites for BMCA include such components and tissue as bones, calcified tissues, cancerous tissues, cell membranes, enzymes, fat, certain fluids (such as spinal fluid), proteins etc. BMCAs used in this invention may also include pharmaceutically accepted salts, esters, and derived compounds thereof, including any organic or inorganic acids or bases. BMCA may be accompanied by other agents, such as salts, oils, fats, waxes, emulsifiers, starches, wetting agents which may be used to aid in carrying the BMCAs to the target more rapidly or more securely, or in diffusing the BMCAs into external tissue such as skin.

During therapy planning prior to actual therapy, it is necessary to account for the thickness of the tissue in the particle pathway before the beam meets the target region. More specifically the relative thickness of fatty tissue, fluids, muscle and connective tissue in the beam pathway needs to be known. This information is conventionally derived from medical imaging (CT, MR, PET, ultrasound) and used in therapy planning algorithms. Yet, because of slightly different patient positioning, or shift/change in state of organs, tissue and fluid as compared to the imaging session, the composition of material in the particle beam pathway may have changed in the therapy session. In some embodiments of the invention, the BMCA signal decrease mechanism can be used for optimizing radiation dosage, energy and/ or duration in order to achieve on-line, real-time therapy optimization. Thus, during irradiation, the parameters of therapy can be modified based upon feedback from the BMCA signals originating from the target tissue.

The following procedure may be applied in order to optimize therapy during the therapy session. A BMCA which can be inactivated by interaction with the particle beam is introduced, and the decrease of signal from the contrast agent is measured as a function of irradiation dose and duration. From experimental measurements, the decrease of contrast agent signal under known conditions is measured and stored in a memory. This may include several tables with different known conditions (e.g. different thicknesses of tissue in the particle beam pathway). The actual decrease of the contrast agent signal is measured during the therapy session, and compared to the stored expected values from the experimental data in the table. From the difference of actual and expected signal values, corrected irradiation parameters (dose, energy, duration) are calculated. The corrected parameters may be output to the operator, or the particle beam controlled automatically.

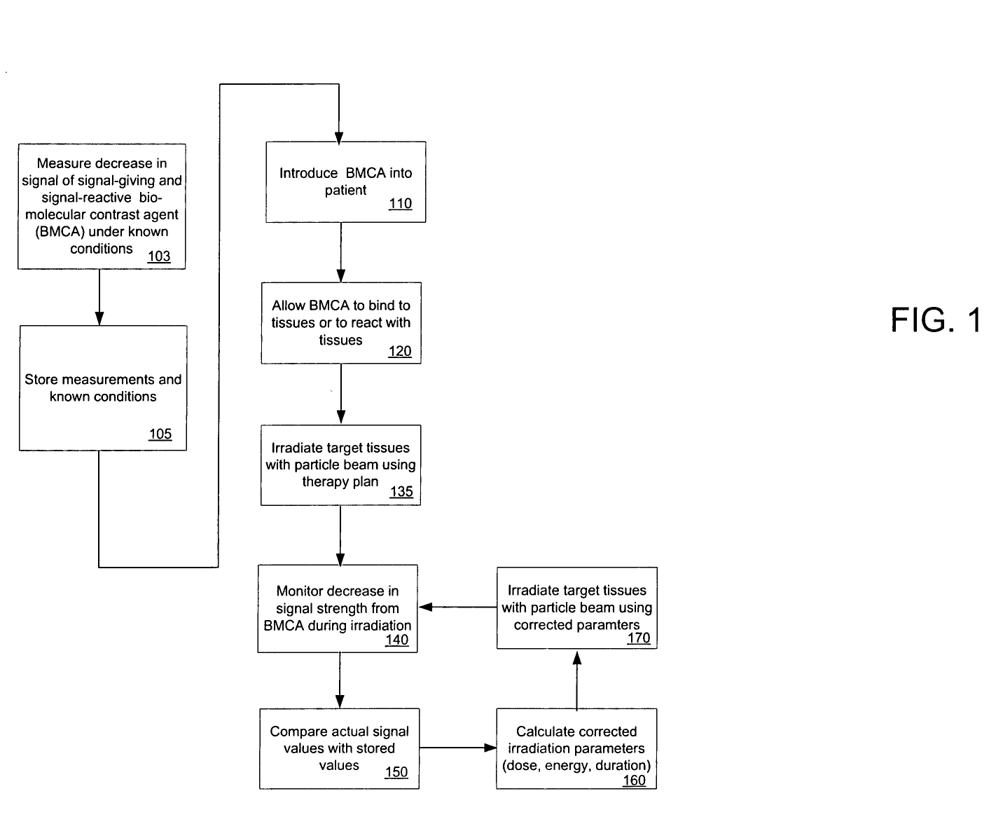

FIG. 1 illustrates therapy optimization using BMCA according to one embodiment of the invention. First, the decrease in signal (strength) of one or more signal-giving and signal-reactive BMCAs is measured under known conditions (block 103). Such known conditions include different thicknesses of tissue, different types of tissue, fluid or other matter, as well as the state of such tissue, fluid and matter found within a patient. These experiments are performed in order to build reference points of how to correlate decrease in signal strength to potentially changing conditions. The experiments can be actual or virtual or theoretical, or a combination of any of these, whichever is most desirable. Further, dynamic models of how BMCAs behave respective to certain conditions can also be built, eliminating the need for static tables. The experiments and/or modeling can be performed such that a wide variety of BMCAs and known conditions can be made available for therapy optimization. The results of these experiments and known conditions are stored in a table, memory or similar mechanism for later use and retrieval (block 105). Alternatively, models that are derived can be programmed or wired into a therapy planning system, if desired.

Blocks 105 and 103 are typically performed once, with the results utilizable to different patients and their respective therapy sessions. In other embodiments of the invention, the blocks 103 and 105 can be repeated for each patient to build a custom set of values for that patient. Once the correlation of values and conditions is established, whether by modeling, experimentation or a combination of these, the patients (or other biological organisms) can be treated by introduction of BMCA (block 110). Methods for introduction of BMCA may be similar to methods used to introduce other contrast agents, such as intravenous or oral and may be targeted or non-specific (such as those which spread throughout a region of the body). Other methods specific to BMCA may also be used. The BMCA, once introduced, is allowed to bind to tissues or react with the tissues (block 120). Thus, a suitable delay after introduction of the BMCA is required. This delay will vary based upon the type of binding or reaction, the type, size and location of the target tissue, the characteristics/affinity of the BMCA, and so on. The time for allowance should be sufficient to stabilize the BMCA binding or reaction with the target.

The BMCAs introduced according to block 110 are also “signal-reactive” in that the strength of signal diminishes with an increasing dose of radiation. This diminution of signal can be proportional to the increase in dosage, duration or other therapy parameters. Alternatively, the BMCA can be designed/selected such that the signal diminishes to zero or almost zero (to an indistinguishable or sensor indistinguishable level) in. If the proportionality is linear, the ratio of decrease in signal strength of the BMCA compared to the variance in therapy parameters should not be too high. A very high ratio would not give meaningful results in terms of measuring therapy parameters. Similarly a very low ratio would not give meaningful results as minute changes in signal may not be able to interpreted accurately.

Next, the target tissues are irradiated with the particle/radiation beam (block 135) which may include any form of radiation including particle beams comprised of one or more of protons, electrons and photons. During irradiation, the strength of the signal from the BMCA is monitored continuously or at defined intervals (block 140). This monitoring may be performed by manual and/or automated means. In either case, a detection/sensing system would capture the strength of the signal and convert this into a value or set of actual signal values. The actual signal values are compared then to the stored values (block 150). Alternatively, the actual signal values can be used to parameterize the models that were built, if any. The comparison process (at block 150) will enable the calculation/computation if corrected therapy parameters. From the difference of actual signal values and stored signal values, the change in conditions can be determined. Utilizing this determined change in conditions, corrected parameters of the therapy such as irradiation parameters of dose, energy, duration and so on, can be calculated (block 160). The corrected therapy parameters are then utilized, either automatically and/or manually, to modify the particle beam and irradiate the target using the corrected parameters (block 170). Any other changing conditions can also be accounted for by continually monitoring the decrease in signal strength from the BMCA (block 140) and correlating and correcting as needed (blocks 150-170).

The difference between actual signal values and stored “expected” signal values will indicate any change in conditions from prior therapy planning. For example, assume during a therapy plan that it was imaged or discovered that the target tissue had a total thickness of X. Given this thickness X, assume further that it was determined that a dosage of Y was required to destroy the target. Assume also that the stored table of expected signal values would correlate a dosage of Y at a thickness X with a decrease in BMCA signal of a value K. If after irradiating the target with the dosage Y of radiation, the actual BMCA signal decrease was only 0.8*K. The expected decrease in signal was not observed and thus, the tissue may have a greater thickness than X. This also implies that the dosage of Y is inadequate and may need to be increased in order to successfully irradiate the target tissue in that direction.

The correlation in the above example relates dosage delivered to the target tissue with an expected signal decrease at a particular thickness of the target tissue. In one embodiment, it may then be possible to match the received actual signal decrease of 0.8*K and match this by doing a reverse look-up or linear regression to find the appropriate required dosage. In this instance, a conclusion could be drawn that the target tissue is thicker in actuality than that measured for therapy planning purposes. For instance, Table I below illustrates a set of possible correlations which can be obtained through experimentation, models and/or study.

TABLE I

| Tissue Thickness | Dosage Delivered | BMCA signal decrease |
|---|---|---|
| X | Y | K |
| X | 2Y | 2K |
| X | 3Y | 3K |
| 2X | Y | 0.8K |
| 2X | 1.5Y | K |
| 2X | 2.5Y | 1.5 * K |

Since a dosage of Y delivered to a tissue of supposed thickness X did not result in the expected signal decrease, then it can be assumed, under this example, that the tissue is thicker than previously believed. The actual signal decrease of 0.8K is observable, under a dosage of Y at a tissue thickness of 2 X. Given that the tissue thickness is 2 X instead of X as previously estimated, the dosage of radiation may have to be increased. This increase can take the form of increased energy and/or increased duration. For instance, it appears that at a thickness of 2 X a dosage of 1.5*Y is needed to achieve a decrease in signal of K. If the signal decrease is also proportional to the level of tumor destruction, then one possible optimization could be to increase the dosage delivered (which is a function of duration and beam energy) to 1.5 Y.

The above example considers only one variable or condition, namely thickness. It is possible for instance that the difference in expected and actual signal decrease is due to a change in another condition, such as proportion of water and fat in the tissue, or due to an obstruction in the path of the beam. The multi-variant nature of the potential conditions and their effect on BMCA signals can be resolved by expanding the number of correlations and cross-correlations, building expert systems or neural networks and by obtaining other data (through imaging and other sensing) to resolve the unknown conditions. Such techniques are well-known and not a subject of the invention. Therapy optimization may involve a large number of potential causes (such as a change in thickness condition) and potential solutions (such as increasing dosage). In accordance with the invention, the use of BMCA can help to resolve these causes and solutions and may be utilized in conjunction with human assistance/interpretation, expert systems, neural networks, models and other mechanisms to derive optimization parameters.

Some potential conditions include the thickness of target tissue, the state of the target tissue, the composition of target tissue, the position/location/extension of target tissue, and the make-up of the pathway through the body to the target tissue. Parameters that can be optimized include the duration of the beam, the energy of the beam, the angle or direction of the beam into the patient, the size of the beam and so on. BMCA signal strength can be tested under these various conditions so that reference points can be established for diagnosing change in conditions or change in therapy parameters. The testing of BMCA signal strength can be established generically for all patients, patient-by-patient or patient type by patient type. In addition, the BMCA reference and conditions data may distinguish different data sets for different types of diseases or patient problems as well.

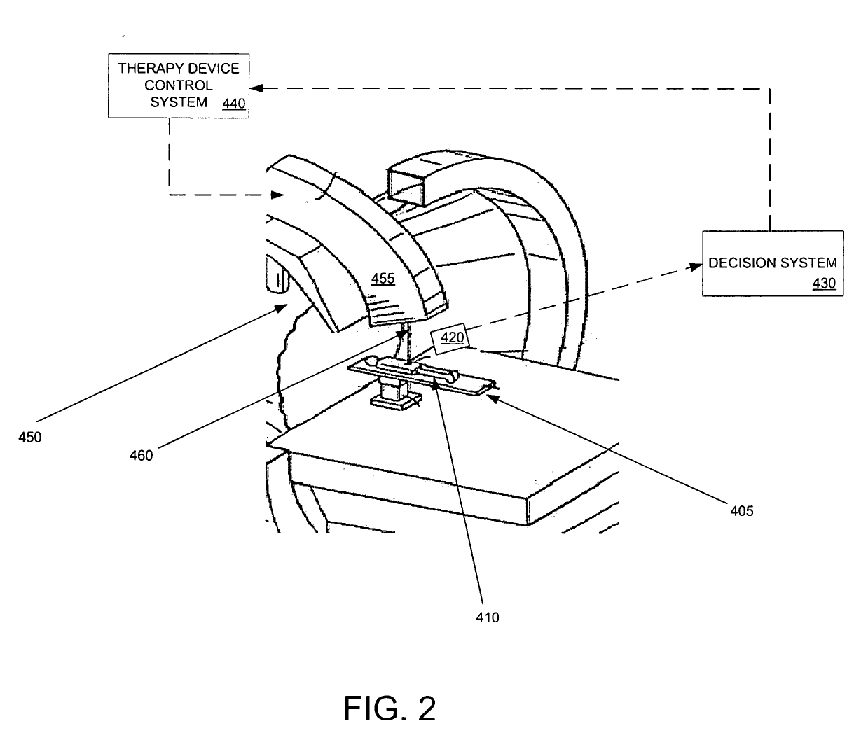

FIG. 2 illustrates a system utilizing one or more embodiments of the invention. At least a portion of a treatment room 400 is shown which houses a therapy device 450 and bed 405 which positions a patient 410 for treatment by treatment device 450. Treatment device 450 may be a radiation or energy delivery system such as proton or photon particle beam delivery system. Treatment device 450 may include a gantry (pictured but not enumerated) and treatment head 455. Treatment head 455 is responsible primarily for delivering and directing the desired or planned energy to patient 410 in the form of a beam 460, for instance. Treatment head 455 may include a number of different elements include scattering elements, collimators, boluses, refraction/reflection elements, and so on.

Generally, in the case of a beam 460 which is composed of particles (such as photons, protons, electrons, neutrons and heavy ions), a particle stream is externally generated and accelerated (by a cyclotron and/or linear accelerator) and then the particle stream (or a portion of it) is delivered to treatment head 455. Treatment head 455 can limit or define both the size and shape of the beam 460 as well as the intensity of the beam 460. Treatment head 455 may also contain a nozzle which can be rotated in different axes to deliver the beam 460. Utilizing this nozzle and various elements within the treatment head 455, therapy device 450 can deliver energy into patient 410 at a different incident angle and with varying shape, size and intensity, as desired. A therapy device control system 440 may be employed for the purpose of controlling the various elements of the treatment head 455 and for controlling the level of energy introduced from the externally generated particle source.

As mentioned above, tables of know conditions, treatments and corresponding BMCA signal strength can be obtained through a combination of experimentation and modeling. This “reference data” may be stored as part of a decision system 430 or stored externally and made available thereto via a network or other communication means. Additional mechanisms such as models, software, neural networks and the like which assist in determining which conditions have changed and how and what the solutions are can be again part of the decision system 430 or separate but accessible thereto.

A therapy plan is ordinarily generated prior to actual therapy beginning. This therapy plan may have been based upon assumptions of certain parameters of the patient 410 such as the size and location of the target tissue, the composition of the target tissue, the composition of the pathway to the target tissue through the body, the state of the tissue and so on. The therapy plan may include parameters such as the geometry and location of the target tissue, marking the body, pre-therapy imaging of the target tissue, dosage plans, and the like. Since the pre-therapy plan is based upon currently available information on target tissue and related conditions, such conditions may change by the time treatment is actually commenced. In addition, it possible that conditions did not necessarily change but were misdiagnosed due to faulty data, faulty interpretation, etc. In such cases as well, the pre-therapy treatment plan may be inaccurate.

In accordance with the invention, prior to treatment by treatment device 450, a BMCA is introduced into patient 410. The BMCA is given time to bind or react to target tissue within the patient 410 to which the beam 460 is to be directed. The therapy device control system 440 utilizes the therapy plan to direct beam 460 towards patient 410. This begins irradiation of the target tissue.

During irradiation, the conditions of the target tissue can be tracked by a sensing system 420. Sensing system 420 will be capable of receiving or detecting the signal emitted by the signal-giving property of the BMCA which is bound to the target tissue within patient 410. Sensing system 420 may be, for example, an optical tomography device or a diaphonoscope which can detect the fluorescence given off the BMCA. The signals emitted by the BMCA may be optical, ultraviolet, infrared, electromagnetic (in the case of a radio-pharmaceutical BMCA), and so on. Sensing system 420 will be designed/selected in order to detect this signal and transfer this sensor data to decision system 430. Sensing system 420 may also include a source (not pictured) such as X-ray source in the case of simple X-ray imaging. Sensing system 420 will be able detect the presence and strength of the BMCA signal emitted from the target tissue within patient 410. This data can be compared against experimental BMCA signal strength data to determine if any conditions of the patient 410 vary from that ascertained in pre-therapy planning. While sensing system 420 is pictured as a non-integrated unit, it can be integrated with the treatment head 455, if desirable, or positioned or integrated anywhere on the therapy device 450 as appropriate.

In some embodiments of the invention, the BMCA signal can be inactivated by exposure to beam 460. In such instances, the sensing system will detect the strength of the BMCA signal as an indication of impaction of beam 460 with the target. In response to data received from sensing system 460, decision system 430 will be configured to determine any change in conditions of the target tissue. Decision system 430 may also have access to a pre-therapy planning data and images of the target tissue, if needed for additional analysis. Decision system 430 will determine if there is a change in conditions of the target tissue based upon differences in actual and experimental BMCA signal values. If there is, and this change is significant enough to affect the outcome of the therapy, or if the change would indicate a change in the therapy plan, then decision system 430 can indicate these changes to the therapy device control system 440. Based upon these changes, the therapy device control system 440 can change the dosage, duration or positioning parameters of the beam 460 to resolve the change or variance conditions of the target tissue. The beam 460 can be also stopped altogether, if necessary, particularly if the sensing system 420 and decision system 430 indicate that the target tissue is no longer present. The decision system 430 may send condition change and/or resolution information to an operator which can then manually implement the modified therapy parameters to the therapy device control system 440 if deemed necessary. In other embodiments of the invention, the changes in operation of the therapy device control system 440 can be automated, whichever is more desired. In other embodiments of the invention, the therapy device control system 440 could modify the position of the patient 410 or the bed 405 in response to decision system 430 indicating a change in conditions of the target tissue.

The systems mentioned in the above description including the sensing system 420, decision system 430 and therapy device control system 440 may be any combination of hardware, software, firmware and the like. Further, all of these systems may be integrated onto the same hardware platform or exist as software modules in a computer system or both. The systems may be distributed in a networked environment as well and may be stand-alone components. One or more of the systems 420, 430 and 440 may be integrated with the therapy device 450 itself, or separate therefrom. Further, any number of these systems 420, 430 and 440 may be physically separated from the therapy device and manually/automatically monitored or controlled. Systems 420, 430 and 440 may utilize or be loaded into processors, storage devices, memories, network devices, communication devices and the like as desired. Sensing system 420 may also contain cameras, sensors, and other active/passive detection and data conversion components, without limitation.

While the embodiments of the invention are illustrated in which it is primarily incorporated within a radiation therapy system, almost any type of medical treatment of imaging system may be potential applications for these embodiments. Further, the bio-molecular contrast agents used in various embodiments may be any organic or semi-organic compounds which have the desired effect of affinity to certain target tissues/cells to either bind with them or react with them. The examples provided are merely illustrative and not intended to be limiting.

1. A method for treating a target with a beam of energy, said target being within a biological organism, said method comprising:
introducing a bio-molecular contrast agent (BMCA) into said biological organism, said BMCA being capable of at least one of binding to said target and reacting with said target, said BMCA capable of also giving detectable signals;
irradiating said target using said beam of energy in accordance with a pre-therapy plan;
after said BMCA has bound or reacted to said target, determining if said BMCA signals indicate that the conditions of said target have varied from said pre-therapy plan;
if deemed necessary optimizing said irradiating of said target in accordance with varied conditions.

2. A method according to claim 1 wherein said target is a tissue in a particular state.

3. A method according to claim 1 further comprising:
sensing of said BMCA signals.

4. A method according to claim 1 wherein said conditions include at least one of tissue state, location of aid target, composition of said target, pathway to said target, and geometry of said target.

5. A method according to claim 3 wherein said sensing is performed using an imaging technique, further said BMCA signals are capable of being imaged.

6. A method according to claim 5 wherein said imaging technique is at least one of optical imaging, positron emission tomography, magnetic resonance imaging, X-ray imaging, ultrasound imaging and computed tomography.

7. A method according to claim 1 wherein said BMCA signals include at least one of fluorescence, luminescence and phosphorescence.

8. A method according to claim 3 wherein determining includes:
comparing said sensed BMCA signals with expected BMCA signals; and
utilizing said comparison to determine variance in conditions, if any.

9. A method according to claim 6 wherein said optical imaging includes detecting at least one of visible, infrared and ultraviolet signals given by said BMCA.

10. A method according to claim 8 wherein said comparing includes determining the difference of said expected and said sensed BMCA signals.

11. A method according to claim 1 wherein said beam of energy is composed at least one of proton, photon, heavy ion, neutron and electron particles.

12. A method according to claim 8 wherein utilizing includes:
correlating said compared BMCA signals with treatment parameters under at least one of said conditions.

13. A method according to claim 1 wherein said optimizing of the irradiation includes at last one of optimizing the energy, dosage and duration of the irradiation.

14. A system for treating a target with a beam of energy from a therapy device, said target within a biological organism, said therapy device initially configured for treating according to a pre-therapy plan, said system comprising:
a sensing system configured to detect signals from bio-molecular contrast agents (BMCA) introduced into said biological organism, said BMCA binding to or reacting with said target;
a decision system configured to compare said sensed BMCA signals with expected BMCA signals and determine any variance in conditions from said pre-therapy plan therefrom; and
a therapy device control system configured to optimize the treating of said target based upon said any variance.

15. A system according to claim 14 wherein said decision system determines if and in what manner said optimizing of treating will be performed.

16. A system according to claim 14 further comprising:
a mechanism for correlating the comparison of said expected and sensed BMCA signal values with treatment parameters for at least one of said conditions.

17. A system according to claim 16 wherein said treatment parameters include dosage of said energy delivered by said treatment device.

18. A system according to claim 14 wherein said treatment device includes a particle therapy device.

19. A system according to claim 18 wherein said particle therapy device is a proton therapy device.

20. A system according to claim 14 wherein said BMCA signals decrease in strength as said beam of energy is treating said target.

21. A system according to claim 14 wherein said conditions include at least one of tissue state, composition of said target, pathway to said target, and geometry of said target.

22. A system according to claim 16 wherein said treatment parameters include duration of said energy delivered by said treatment device.

23. A system according to claim 16 wherein said treatment parameters include the energy level of said energy delivered by said treatment device.
